(19) United States
(12) Patent Application Publication
(10) Pub. No.: US 2023/0376648 A1
(43) Pub. Date: Nov. 23, 2023

(54) HYBRID QUANTUM-CLASSICAL COMPUTING SYSTEM FOR SIMULATION OF CHEMICAL SYSTEMS USING A CHEMICALLY AWARE STATE-PREPARATION STRATEGY

(71) Applicant: Cambridge Quantum Computing Limited, London (GB)

(72) Inventors: Irfan Khan, Cambridge (GB); Josh Kirsopp, Leeds (GB)

(21) Appl. No.: 18/048,147

(22) Filed: Oct. 20, 2022

Related U.S. Application Data
(60) Provisional application No. 63/344,629, filed on May 22, 2022.

(51) Int. Cl. G06F 30/20

(52) U.S. Cl. CPC G06F 30/20

(57) ABSTRACT

A chemical system is simulated using a hybrid quantum-classical computing system. The hybrid quantum-classical computing system comprises a classical computing component coupled to a quantum computing component. The hybrid quantum-classical computing system is configured to determine characterizing parameters that describe the chemical system, wherein the characterizing parameters are at least partially based on a chemical structure of the chemical system; generate a operator representation of the chemical system, including single electron excitation operators and double electron excitation operators; and reconfigure an order of occurrence of the single electron excitation operators and the double electron excitation operators in the operator representation as a function of the characterizing parameters. The simulation is executable with a lower count of two-qubit quantum gates used in at least one quantum circuit executable on the quantum computing component that is used to implement the simulation.

### CROSS-REFERENCE TO RELATED APPLICATIONS

This application claims priority to U.S. Application No. 63/344,629, filed May 22, 2022, the content of which is hereby incorporated herein by reference in its entirety.

### TECHNICAL FIELD

The present disclosure relates to computing systems for simulating chemical systems, and method for using such computing systems for simulating chemical systems. An example embodiment relates to the use of hybrid quantum-classical computing systems for simulating chemical systems.

### BACKGROUND

A given chemical system, including, but not limited to, an atom; a molecule; an ion; a periodic solid; a periodic surface slab; a collection of one or more atoms, molecules, and/or ions; or a combination of these, consists of one or more atomic nuclei and at least one electron. The quantum state of said at least one electron is represented by an electronic wavefunction, which is conveniently expressed as a linear combination of Slater determinants, built from molecular (or crystal) orbitals.

As the number of electrons in an atom and/or atoms in a molecule, periodic solid, and/or periodic surface slab increases, the computational complexity and processing costs of modeling such systems increases significantly, for example substantially exponentially. Through applied effort, ingenuity, and innovation many deficiencies of modeling and/or simulating such systems have been solved by developing solutions that are structured in accordance with the embodiments of the present invention, many examples of which are described in detail herein.

### BRIEF SUMMARY OF EXAMPLE EMBODIMENTS

Various embodiments provide methods, systems, apparatus, computer program products, and/or the like for simulating chemical systems. Example embodiments provide methods, systems, apparatus, computer program products, and/or the like for simulating chemical systems that are capable of simulating chemical systems such as atoms; molecules; ions; periodic solids; periodic surface slabs; collections of one or more atoms, molecules, and/or ions; or combinations thereof, using a quantum computer through the execution of a reduced depth quantum circuit.

In various embodiments, a hybrid quantum-classical computing technique is used to accurately and efficiently model and/or simulate chemical systems. In particular, at least one quantum circuit encoding the chemical system simulation is generated based on the transformation of a parameterized representation and/or operator representation of the chemical system from the fermionic Hilbert space to the qubit Hilbert space for qubits of a quantum computing component to be used to execute the at least one quantum circuit. The depth (e.g., number of quantum gates and/or layers of quantum gates) of the at least one quantum circuit is reduced based on characterizing parameters of the chemical system and/or through the reordering of the occurrence of excitation operators in the operator representation of the chemical system. For example, in an example embodiment, the at least one quantum circuit is configured to cause the performance of excitation operators in the order of double excitation operators corresponding to double excitations from a spatial orbital to a corresponding spatial orbital, arbitrary double excitation operators (e.g., corresponding to double electron excitations other than those from a spatial orbital to a corresponding spatial orbital), and single electron excitation operators.

In various embodiments, the arbitrary double excitation operators are Jordan-Wigner encoded arbitrary double excitation operators configured to act on 4-qubits or more (e.g., four or more qubits).

In various embodiments, the number of excitation operators in the operator representation of the chemical system are reduced based on symmetries of the chemical system. For example, excitations that are redundant due to the symmetry of the chemical system and/or excitations that are not allowed based on the symmetry of the chemical system are filtered out of the operator representation to decrease the number of gates required to perform the resulting quantum circuit. In various embodiments, a reduced depth quantum circuit is determined and/or generated that, when executed by the quantum computing component of a hybrid quantum-classical computing component, determines a wavefunction of the chemical system, how the chemical system behaves in one or more interactions (e.g., with other chemical systems and/or with electromagnetic radiation), determine a structural characteristic of the chemical system, and/or the like.

According to a first aspect, a hybrid quantum-classical computing system configured for implementing a simulation of a chemical system is provided. The hybrid quantum-classical computing system comprises a classical computing component coupled to a quantum computing component. The hybrid quantum-classical computing system is configured to determine characterizing parameters that describe the chemical system, wherein the characterizing parameters are at least partially based on a chemical structure of the chemical system; generate a operator representation of the chemical system, including single electron excitation operators and double electron excitation operators; and reconfigure an order of occurrence of the single electron excitation operators and the double electron excitation operators in the operator representation as a function of the characterizing parameters. The simulation is executable with a lower count of two-qubit quantum gates used in at least one quantum circuit executable on the quantum computing component that is used to implement the simulation.

In an example embodiment, the hybrid quantum-classical computing system is configured to reconfigure the order of occurrence of the single electron excitation operators and the double electron excitation operators to (i) double excitation operators corresponding to double excitations from a spatial orbital to a corresponding spatial orbital; (ii) arbitrary double excitation operators; and (iii) arbitrary single excitation operators.

In an example embodiment, the hybrid quantum-classical computing system is configured to determine the characterizing parameters that describe the chemical system and to generate a corresponding operator representation by applying symmetry filtering to the operator representation of the chemical system to assess its structural degree of symmetry; to generate a quantum state synthesis of the chemical system based on the degree of symmetry; to generate a spatial excitation synthesis of the chemical system from the quantum state synthesis; to process the spatial excitation synthesis by transforming from spatial orbitals to spin orbitals therein; to introduce spin orbitals on the at least one quantum circuit; and use commuting sets of double excitation operators and single excitation operators to synthesize the at least one quantum circuit, such that double excitation operators appear in the at least one quantum circuit before the single excitation operators appear in the at least one quantum circuit and each commuting set corresponds to an excitation of the chemical system.

In an example embodiment, the hybrid quantum-chemical computing system is configured to reduce a number of quantum gates required in the quantum circuit synthesis as a function of the structural degree of symmetry of the chemical system.

In an example embodiment, the hybrid quantum-chemical computing system is configured to apply the symmetry filtering to the operator representation comprising at least one of defining a set of symmetries of the chemical system and identifying excitation operators of the operator representation of the chemical system that do not commute with one or more symmetries of the set of symmetries, or defining an abelian point group of the chemical system and identifying redundant excitations of the chemical system based on the abelian point group.

In an example embodiment, generating the quantum state synthesis of the chemical system comprises generating a (double occupation of spatial orbitals) reference state based on the double occupied and virtual spatial orbitals of the chemical system.

In an example embodiment, the spatial excitation synthesis comprises transforming spatial orbital to spatial orbital operators to double excitation spatial-to-spatial qubit operators.

In an example embodiment, generating the quantum circuit synthesis comprises transforming the double excitation operators (e.g., the arbitrary double excitation operators) of the chemical system that correspond to double excitations other than spatial orbital to spatial orbital double excitations to arbitrary double excitation qubit operators.

In an example embodiment, the hybrid quantum-classical computing entity is configured to compile the quantum circuit synthesis into an executable quantum circuit, wherein the quantum circuit synthesis comprises, in order, the double excitation spatial-to-spatial qubit operators, the arbitrary double excitation qubit operators, and single excitation qubit operators.

In an example embodiment, the hybrid quantum-classical computing system is further configured to cause the quantum computing component to execute the at least one quantum circuit to determine at least one of a wavefunction of the chemical system, structural characteristic of the chemical system, a chemical interaction characteristic of the chemical system, or a response characteristic of the chemical system.

In an example embodiment, the hybrid quantum-classical computing system is further configured to cause the classical computing component to cause at least one of (a) a display of a graphical representation of at least a portion of the simulation of the chemical system or (b) a generation and storage in a classical memory of a file comprising one or more parameters of the simulation of the chemical system.

In an example embodiment, the operator representation is generated using a unitary coupled cluster singles and doubles (UCCSD) ansatz.

According to another aspect, a method for using a hybrid quantum-classical computing system to implement a simulation of a chemical system is provided. The hybrid quantum-classical computing system comprises a classical computing component coupled to a quantum computing component. In an example embodiment, the method comprises determining characterizing parameters that describe the chemical system, wherein the characterizing parameters are at least partially based on a chemical structure of the chemical system; generating a operator representation of the chemical system, including single electron excitation operators and double electron excitation operators; and reconfiguring an order of occurrence of the single electron excitation operators and the double electron excitation operators in the operator representation as a function of the characterizing parameters. The simulation is executable with a lower count of two-qubit quantum gates used in at least one quantum circuit executable on the quantum computing component that is used to implement the simulation.

In an example embodiment, the order of occurrence of the single electron excitation operators and the double electron excitation operators is reconfigured to (i) double excitation operators corresponding to double excitations from a spatial orbital to a corresponding spatial orbital; (ii) arbitrary double excitation operators; and (iii) arbitrary single excitation operators.

In an example embodiment, the system is configured to determine the characterizing parameters that describe the chemical system and to generate a corresponding operator representation by applying symmetry filtering to the operator representation of the chemical system to assess its structural degree of symmetry; generating a quantum state synthesis of the chemical system based on the degree of symmetry; generating a spatial excitation synthesis of the chemical system from the quantum state synthesis; processing the spatial excitation synthesis by transforming from spatial orbitals to spin orbitals therein; introducing spin orbitals on the at least one quantum circuit; and using commuting sets of double excitation operators and single excitation operators to synthesize the at least one quantum circuit, such that double excitation operators appear in the at least one quantum circuit before the single excitation operators appear in the at least one quantum circuit and each commuting set corresponds to an excitation of the chemical system.

In an example embodiment, the hybrid quantum-chemical computing system is configured to reduce a number of quantum gates required in the quantum circuit synthesis as a function of the structural degree of symmetry of the chemical system.

In an example embodiment, applying the symmetry filtering to the operator representation comprises at least one of defining a set of symmetries of the chemical system and identifying excitation operators of the operator representation of the chemical system that do not commute with one or more symmetries of the set of symmetries, or defining an abelian point group of the chemical system and identifying redundant excitations of the chemical system based on the abelian point group.

In an example embodiment, generating the quantum state synthesis of the chemical system comprises generating a (double occupation of spatial orbitals) reference state based on the double occupied and virtual spatial orbitals of the chemical system.

In an example embodiment, the spatial excitation synthesis comprises transforming spatial orbital to spatial orbital operators to double excitation spatial-to-spatial qubit operators.

In an example embodiment, generating the quantum circuit synthesis comprises transforming the double excitation operators of the chemical system that correspond to double excitations other than spatial orbital to spatial orbital double excitations (e.g., the arbitrary double excitation operators) to arbitrary double excitation qubit operators.

In an example embodiment, the method further comprises compiling the quantum circuit synthesis into an executable quantum circuit, wherein the quantum circuit synthesis comprises, in order, the double excitation spatial-to-spatial qubit operators, the arbitrary double excitation qubit operators, and single excitation qubit operators.

In an example embodiment, the method further comprises causing the quantum computing component to execute the at least one quantum circuit to determine at least one of a wavefunction of the chemical system, a structural characteristic of the chemical system, a chemical interaction characteristic of the chemical system, or a response characteristic of the chemical system.

In an example embodiment, the method further comprises causing the classical computing component to cause at least one of (a) display of a graphical representation of at least a portion of the simulation of the chemical system or (b) generation and storage in a classical memory of a file comprising one or more parameters of the simulation of the chemical system.

In an example embodiment, the operator representation is generated using a unitary coupled cluster singles and doubles (UCCSD) ansatz.

According to another aspect a computer program product is provided. In an example embodiment, the computer program product comprises at least one non-transitory computer-readable medium storing executable instructions. The executable instructions are configured to, when executed by a hybrid quantum-classical computing system, cause the hybrid quantum-classical computing system to perform determining characterizing parameters that describe the chemical system, wherein the characterizing parameters are at least partially based on a chemical structure of the chemical system; generating a operator representation of the chemical system, including single electron excitation operators and double electron excitation operators; and reconfiguring an order of occurrence of the single electron excitation operators and the double electron excitation operators in the operator representation as a function of the characterizing parameters, such that the simulation is executable with a lower count of two-qubit quantum gates used in at least one quantum circuit executable on the quantum computing component that is used to implement the simulation.

In an example embodiment, the order of occurrence of the single electron excitation operators and the double electron excitation operators is reconfigured to (i) double excitation operators corresponding to excitations from a spatial orbital to a corresponding spatial orbital; (ii) arbitrary double excitation operators; and (iii) arbitrary single excitation operators.

In an example embodiment, the executable instructions are further configured, when executed by the hybrid quantum-classical computing system, to cause the hybrid quantum-classical computing system to determine the characterizing parameters that describe the chemical system and generate a corresponding operator representation by applying symmetry filtering to the operator representation of the chemical system to assess its structural degree of symmetry; generating a quantum state synthesis of the chemical system based on the degree of symmetry; generating a spatial excitation synthesis of the chemical system from the quantum state synthesis; processing the spatial excitation synthesis by transforming from spatial orbitals to spin orbitals therein; generating a quantum circuit synthesis by commuting sets of double electron excitation operators from the processed spatial excitation synthesis; and amending the quantum circuit synthesis by commuting sets of singles, such that double excitation operators appear in the quantum circuit synthesis before the single excitation operators appear in the quantum circuit synthesis.

In an example embodiment, the executable instructions are further configured, when executed by the hybrid quantum-classical computing system, to cause the hybrid quantum-classical computing system to reduce a number of quantum gates required in the quantum circuit synthesis as a function of the structural degree of symmetry of the chemical system.

In an example embodiment, applying the symmetry filtering to the operator representation comprises at least one of defining a set of symmetries of the chemical system and identifying excitation operators of the operator representation of the chemical system that do not commute with one or more symmetries of the set of symmetries, or defining an abelian point group of the chemical system and identifying redundant excitations of the chemical system based on the abelian point group.

In an example embodiment, generating the quantum state synthesis of the chemical system comprises generating a (double occupation of spatial orbitals) reference state based on the double occupied and virtual spatial orbitals of the chemical system.

In an example embodiment, the spatial excitation synthesis comprises transforming spatial orbital to spatial orbital operators to double excitation spatial-to-spatial qubit operators.

In an example embodiment, generating the quantum circuit synthesis comprises transforming the double excitation operators of the chemical system that correspond to double excitations other than spatial orbital to spatial orbital double excitations (e.g., arbitrary double excitations) to arbitrary double excitation qubit operator operators.

In an example embodiment, the executable instructions are further configured, when executed by the hybrid quantum-classical computing system, to cause the hybrid quantum-classical computing system to compile the quantum circuit synthesis into an executable quantum circuit, wherein the quantum circuit synthesis comprises, in order, the double excitation spatial-to-spatial qubit operators, the arbitrary double excitation qubit operators, and single excitation qubit operators.

In an example embodiment, the executable instructions are further configured, when executed by the hybrid quantum-classical computing system, to cause the hybrid quantum-classical computing system to cause the quantum computing component to execute the at least one quantum circuit to determine at least one of wavefunction of the chemical system, a structural characteristic of the chemical system, a chemical interaction characteristic of the chemical system, or a response characteristic of the chemical system.

In an example embodiment, the executable instructions are further configured, when executed by the hybrid quantum-classical computing system, to cause the hybrid quantum-classical computing system to cause the classical computing component to cause at least one of (a) display of a graphical representation of at least a portion of the simulation of the chemical system or (b) generation and storage in a classical memory of a file comprising one or more parameters of the simulation of the chemical system.

In an example embodiment, the operator representation is generated using a unitary coupled cluster singles and doubles (UCCSD) ansatz.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a schematic diagram illustrating an example hybrid quantum-classical computing system, according to an example embodiment.

FIG. 2 is a flowchart illustrating processes, procedures, and/or operations performed by a hybrid quantum-classical computing system to provide an output of a simulation of a chemical system, according to an example embodiment.

FIG. 3 is a flowchart illustrating processes, procedures, and/or operations performed by the hybrid quantum-classical computing system to generate the quantum circuit synthesis for compiling to form a reduced depth quantum circuit, according to an example embodiment.

FIG. 4 provides a schematic diagram of an example controller of a quantum component of a hybrid-classical computing system that is configured to control operation of one or more elements of the quantum component, according to various embodiments.

FIG. 5 provides a schematic diagram of an example classical component of a hybrid quantum-classical computing system that may be used in accordance with an example embodiment.

FIG. 6 provides a plot illustrating a comparison of the number of gates in quantum circuits used to simulate various chemical systems using an example embodiment of the present disclosure, using a commuting sets technique, and using a naïve technique.

FIG. 7 provides a set of plots illustrating a comparison of the number of gates in quantum circuits and improvement in relative error on ground state energy determined based on the quantum circuits using an example embodiment of the present disclosure and using a commuting sets technique.

### DETAILED DESCRIPTION OF SOME EXAMPLE EMBODIMENTS

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. The term “or” (also denoted “/”) is used herein in both the alternative and conjunctive sense, unless otherwise indicated. The terms “illustrative” and “exemplary” are used to be examples with no indication of quality level. The terms “generally,” “substantially,” and “approximately” refer to within engineering and/or manufacturing tolerances and/or within user measurement capabilities, unless otherwise indicated. Like numbers refer to like elements throughout.

Various embodiments provide methods, systems, apparatus, computer program products, and/or the like for simulating chemical systems. Example embodiments provide methods, systems, apparatus, computer program products, and/or the like for simulating chemical systems using reduced depth quantum circuits and/or quantum circuits that include fewer two-qubit quantum gates compared to conventionally generated quantum circuits for simulating the chemical system.

In general, it is expected that quantum computers will enable accurate modeling and/or simulation of chemical systems. For example, it is expected that quantum computers will enable the accurate modeling and/or simulation of chemical systems that are too complex for conventional classical approaches. However, currently operational quantum computing components tend to include relatively low numbers of qubits (e.g., less than 100 qubits) and tend to be relatively noisy. As the depth (e.g., the number of layers of quantum gats) and/or the number of two-qubit quantum gates of a quantum circuit increases, so does the effect of noise on the resulting model or simulation. For conventional techniques for generating quantum circuits to model and/or simulate a chemical system, the depth of the quantum circuit scales as O(n⁴), where n is the number of spin orbitals included in the model and/or simulation. These conventional techniques lead to quantum circuits that include thousands of two-qubit quantum gates, which is clearly beyond the capability of currently operable quantum computing components. Thus, a technical problem exists regarding how to generate quantum circuits for modeling and/or simulating chemical systems that can be executed by currently operable quantum computing components, namely noisy intermediate-scale quantum (NISQ) devices.

Various embodiments provide technical solutions to these technical problems. In particular, various embodiments use the symmetries of the chemical system to reduce the number of two-qubit quantum gates required to accurately model and/or simulate the chemical system. In various embodiments, the number of two-qubit quantum gates of the resulting quantum circuit is at least a factor of ten smaller than a quantum circuit for modeling and/or simulating the same chemical system generated through conventional techniques. Thus, various embodiments provide improvements to the fields of chemical system modeling and/or simulation, quantum chemistry, and/or quantum circuit generation.

In general, a quantum circuit is an ordered sequence of quantum operations (e.g., quantum operators applied to the quantum data of one or more qubits through single or two qubit gates) to be performed on a set of qubits. A quantum computing component can execute a compiled quantum circuit to effect a quantum computation. In various scenarios, a quantum circuit includes layers (i.e. a temporal sequence) of quantum operations where each layer includes a collection of quantum operations that are performed at a respective time step of the quantum computation. The depth and/or number of layers or count of layers in the quantum circuit therefore provides an indication of time length required to perform the quantum computation and/or the number of quantum operations performed on the individual qubits—both of which may affect the amplitude of noise in the result of a respective quantum computation.

In various embodiments, a hybrid quantum-classical computing technique is used to accurately and efficiently model and/or simulate chemical systems. In various embodiments, a wavefunction of the chemical system, a structural characteristic of the chemical system, a chemical interaction characteristic of the chemical system, and/or a response characteristic of the chemical system is determined by executing a quantum circuit via a quantum computing component of the hybrid quantum-classical computing system. In various embodiments, executing the quantum circuit via the quantum computing component causes the quantum computing component to model and/or simulate the chemical system. In various embodiments, the model and/or simulation is a variational quantum eigen solver (VQE) model and/or simulation of the chemical system.

In various embodiments, the generation and/or simulation of the quantum circuit includes determining characterizing parameters that describe the chemical system. The characterizing parameters are determined at least in part based on the chemical structure of the chemical system. For example, the characterizing parameters include nuclei information for each atomic nucleus of the chemical system (e.g., numbers of protons and neutrons present in the respective atomic nuclei), a number of electrons in the chemical system, Cartesian coordinates of the nuclei, various symmetries of the chemical system (e.g., including but not limited to [symbol] ₂ symmetries), spatial and/or spin orbitals of the chemical system (e.g., approximated via Hartree-Fock (HF) techniques and/or other techniques), and/or other parameters that characterize the chemical system, in various embodiments.

In various embodiments, the generation and/or simulation of the quantum circuit includes generating a parameterized representation of the chemical system. In various embodiments, the parameterized representation of the chemical system is a wavefunction of the chemical system. In various embodiments, the parameterized representation of the chemical system is determined based on an operator representation of the chemical system. For example, the parameterized representation is an eigen state of the operator representation of the chemical system, in an example embodiment. In various embodiments, the operator representation of the chemical system is an effective Hamiltonian of at least a portion of the chemical system. In various embodiments, the operator representation is and/or includes an UCC operator (e.g., a UCCSD operator). In various embodiments, the operator representation comprises a plurality of operators configured to operate on various spatial and/or spin orbitals of the chemical system. As should be understood, an operator acting on a first spatial and/or spin orbital may cause a change in the probability that the first spatial and/or spin orbital and/or one or more second spatial and/or spin orbitals of the chemical system are occupied.

In various embodiments, the parameterization of the parameterized representation of the chemical system is determined based on an ansatz to be used to represent the waveform of the chemical system and/or the operators aggregated to form the operator representation of the chemical system.

As should be understood, an operator is a function that acts on an element of a space to produce elements of the same or another space. For example, excitation operators within the Hilbert space of the electrons of the chemical system are configured to act upon a representation of the electrons of the chemical system to cause one or more excitations thereof. An excitation operator that has been transformed and/or mapped to the Hilbert space of the qubits of the quantum computing component acts on the quantum state of a qubit to cause the quantum state of the qubit to represent, model, and/or simulate a feature (e.g., orbital occupation) of the chemical system.

In various embodiments, a unitary coupled cluster (UCC) ansatz is used to represent the waveform of the chemical system and/or operators (e.g., the operators that are aggregated to provide the operator representation) of the chemical system. In an example embodiment, the ansatz is a UCCSD ansatz which is a UCC ansatz that includes single electron excitation operators and double electron excitation operators.

In various embodiments, the electron correlations of the chemical system are modelled and/or simulated using an operator representation of the chemical system (e.g., an effective Hamiltonian of at least a portion of the chemical system). In various embodiments, the basis and/or format of the operator representation is determined based on the ansatz used. For example, in various embodiments using a UCCSD ansatz, the electron correlations are simulated by using a UCCSD operator. For example, in an example embodiment, the parameterized representation of the chemical system is generated based on the operator representation of the chemical system (a UCCSD operator or another ansatz-determined operator).

In an example embodiment, the operator representation is represented as a Trotterized product of exponentials. As is generally understood in the art, Trotterizing a product of exponents is a truncation of the infinite series used to evaluate the product of exponentials. For example, in an example embodiment where the operator representation includes a UCCSD operator, the operator representation is written as e^{\hat{⬆}-\hat{⬆}†}, where \hat{T} is the sum over each excitation operator acting across all of the spin and/or spatial orbitals of the chemical system and \hat{T}^{†} is the Hermitian adjoint thereof. In various embodiments, the excitation operators included in the operator representation (e.g., an ansatz-based representation of the effective Hamiltonian of at least a portion of the chemical system) used to determine and/or generate the parameterized representation of the chemical system are determined based at least in part on one or more of spin-multiplicity of the chemical system, number of electrons in the chemical system, and the basis-set size of the chemical system. In various embodiments, the basis set defines the functional basis used to expand the spin orbitals and is determined, at least in part, based on the ansatz. The basis-set size is half the size of the number of spin orbitals or equal to the number of spatial orbitals, for a restricted wavefunction (e.g., a wavefunction truncated to only include a defined and/or truncated set of spin and/or spatial orbitals).

In an example embodiment, the parameterized representation of the chemical system (e.g., a wavefunction of the chemical system determined and/or generated based on the operator representation of the chemical system, in an example embodiment) and/or the operator representation (e.g., the effective Hamiltonian of at least a portion of the chemical system) is transformed and/or mapped from the Hilbert space of the chemical system to the Hilbert space of the qubits of the quantum computing component. In various embodiments, the transformation and/or mapping of the parameterized representation and/or operator representation of the chemical system from fermionic operators to qubit operators is determined and/or dependent on the type of qubit used by the quantum component of the hybrid quantum-classical computing system being used. For example, the quantum component may use photons, electrons, atomic nuclei, neutral atoms, ions, Josephson junctions, quantum dots, topological anyons, and/or other quantum particles and/or systems as qubits. In an example embodiment, the transformation and/or mapping is performed using a Jordan-Wigner encoding, although various other encodings are used in various other embodiments.

In embodiments using a Jordan-Wigner encoding, for example, each qubit of the quantum circuit represents the electron number occupation of each spin-orbital in the chemical system. For example, when a qubit is in the 11> state, the corresponding spin-orbital is occupied and when the qubit is in the 10> state, the corresponding spin-orbital is not occupied. Using the Jordan-Wigner encoding, the operator representation, which includes a UCCSD operator in an example embodiment, is written as a product of Pauli exponentials such that e^{\hat{⬆}-\hat{⬆}†}≈Πₘ Πₙ e^{iθ}^{ₘ}^{\hat{P}}^{_{m,n}}, where the index m runs over each distinct Fermionic excitation of the chemical system, the index n runs over each Pauli-sub-term \hat{P}_{m,n} of the Pauli operator \hat{P}, and the θₘ, are independent real parameters.

In various embodiments, the generation and/or simulation of the quantum circuit includes reconfiguring the order of occurrence of the single electron excitation operators and the double electron excitation operators in the operator representation (e.g., the effective Hamiltonian of at least a portion of the chemical system) of the chemical system. In various embodiments, the order of the occurrence of the single electron excitation operators and the double electron excitation operators in the operator representation (e.g., the effective Hamiltonian of at least a portion of the chemical system) of the chemical system are reconfigured. The reconfiguration reduces the number and/or lowers the count of two-qubit quantum gates (e.g., two qubit gates such as controlled not gates (CX), ZZ-max gates, iSWAP gates, and/or the like) used in the quantum circuit to model and/or simulate the chemical system. In various embodiments, the reconfiguration of the occurrence of the single electron excitation operators and the double electron excitation operators is performed as a function of the characterizing parameters of the chemical system. For example, the knowledge of the symmetry of the chemical system, spin and/or spatial orbitals of the chemical system, and/or the like are used to reconfigure the order of the occurrence of the single electron excitation operators and the double electron excitation operators in the operator representation (e.g., the effective Hamiltonian of at least a portion of the chemical system) of the chemical system so as to lower the count of two-qubit quantum gates in the resulting quantum circuit.

A quantum circuit is compiled based on the operator representation (e.g., the effective Hamiltonian of at least a portion of the chemical system) of the chemical system after the transformation and/or mapping thereof to qubit operators and the reconfiguring of the order of occurrence of the single and double electron excitation operators. Using a quantum computing component, the quantum circuit is executed to model and/or simulate the chemical system. Measurement operations are performed by the quantum computing component (e.g., as part of the quantum circuit) to extract the qubit representation of chemical system, in an example embodiment.

The qubit representation of the chemical system is then processed to determine one or more of a wavefunction of the chemical system, characteristics indicating how the chemical system behaves in one or more interactions (e.g., with other chemical systems and/or electromagnetic radiation), structural characteristics of the chemical system, and/or the like.

As used herein, a classical computing component or classical computer is a computing entity that uses semiconductor-based computational techniques and hardware. A quantum computing component or quantum computer uses the quantum states of quantum particles (referred to as qubits) to perform computations.

### Example Hybrid Quantum-Classical Computing System

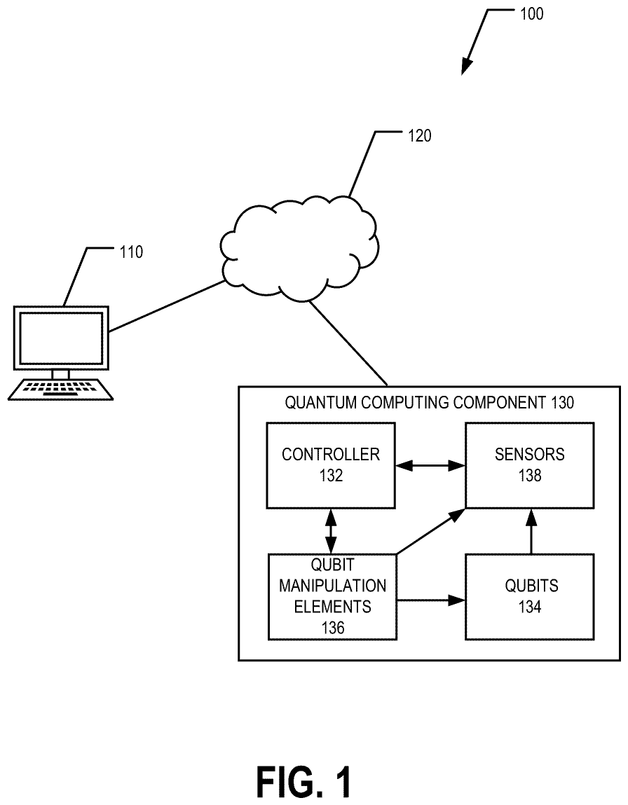

FIG. 1 provides a block diagram of an example hybrid quantum-classical computing system 100, in accordance with various embodiments. In various embodiments, the hybrid quantum-classical computing system 100 comprises a classical component such as a classical computing component 110 and a quantum component such as a quantum computing component 130.

The quantum computing component 130 comprises a controller 132, qubits 134, qubit manipulation elements 136 and sensors 138. The controller 132 is configured to control operation of the qubit manipulation elements 136 to cause desired manipulations (e.g., controlled quantum state evolution) of the qubits 134. The controller 132 is further configured to control operation of the sensors 138 that are configured to monitor, measure, and/or capture measurements corresponding to the operation of the qubit manipulation components and capture measurements indicating the respective quantum states of respective qubits 134.

For example, in various embodiments, the qubit manipulation elements 136 comprise voltage/current sources, laser sources, magnetic field sources (e.g., electromagnets and/or permanent magnets) and/or other hardware components configured for use in confining the qubits and/or manipulating the quantum state of the qubits. For example, in various embodiments, the sensors 138 comprise photodetectors, voltage/current sensors, temperature sensors, pressure sensors, and/or other sensors that may be used to determine a quantum state of a qubit and/or monitor operation of one or more of the qubit manipulation elements 136.

In various embodiments, the classical computing component 110 is in communication with the controller 132 of the quantum computing component 130 via one or more wired or wireless networks 120 and/or via direct wired and/or wireless communications. For example, the classical computing component 110 is configured to generate and provide a quantum circuit configured to model and/or simulate a chemical system to the quantum computing component 130 (e.g., the controller 132 thereof) and receive a qubit representation of the chemical system provided by the quantum computing component 130 (e.g., the controller 132 thereof) via the one or more wired or wireless networks 120 and/or via direct wired and/or wireless communications between the classical computing component 110 and the quantum computing component 130. For example, the quantum computing component 130 is configured to receive a quantum circuit provided by the classical computing component 110 and provide a qubit representation of the chemical system for receipt by the classical computing component 110 via the one or more wired or wireless networks 120 and/or via direct wired and/or wireless communications between the classical computing component 110 and the quantum computing component 130.

In various embodiments, the hybrid quantum-classical computing system 100 and/or portions thereof (e.g., classical computing component 110 and/or quantum computing component 130) are configured to use the InQuanto quantum chemistry application library and/or tket software development kit to perform various process, operations, and/or the like described herein. As should be understood, various other quantum chemistry application libraries and/or software development kits may be used in various other embodiments.

### Example Operation of a Hybrid Quantum-Classical Computing System

In various embodiments, a hybrid quantum-classical computing system 100 is used to simulate a chemical system. For example, the hybrid quantum-classical computing system 100 is used to generate a model of a chemical system that represents a wavefunction of the chemical system, one or more structural characteristics of the chemical system, one or more chemical interaction characteristics of the chemical system, one or more response characteristics of the chemical system, and/or the like, in various embodiments. In various embodiments, the model, a portion thereof, and/or a graphical representation thereof is displayed via a display (e.g., of the classical computing component 110), stored to a file that may be used as an input and/or provided directly as input to other simulations or models that use the interaction, structural characteristics of the chemical system, and/or response characteristics of the chemical system to perform one or more functions thereof, and/or the like.

In various embodiments, the classical computing component 110 obtains information corresponding to a chemical system. For example, user input (e.g., received via a user input interface of the classical computing component 110) may provide, select, and/or cause the accessing of information corresponding to a chemical system. In an example embodiment, the information corresponding to the chemical system may be a chemical formula for the chemical system (H, H₂O, NH₄⁺, OH, CH₄, a specified transition state of a chemical reaction, etc.) and/or other designation of the chemical system. In an example embodiment, the information corresponding to the chemical system includes nuclei information for each atomic nucleus of the chemical system (e.g., numbers of protons and neutrons present in the respective atomic nuclei), a number of electrons in the chemical system, Cartesian coordinates of the nuclei, and/or any other information used to define the chemical system.

In various embodiments, obtaining of the information corresponding to the chemical system triggers and/or causes the classical computing component 110 to determine and/or generate a model of the chemical system that represents a (fermionic) wavefunction of the chemical system, one or more structural characteristics of the chemical system, one or more chemical interaction characteristics of the chemical system, one or more response characteristics of the chemical system, and/or the like.

For example, the classical computing component 110 may determine and/or identify characterizing parameters of the chemical system, use the characterizing parameters to perform a quantum circuit synthesis, and provide the quantum circuit to the quantum computing component 130. The quantum computing component 130 may execute the quantum circuit (e.g., using a plurality of qubits thereof) to determine a qubit representation of the chemical system. For example, in various embodiments, the qubit representation may provide orbital occupation information for a plurality of orbitals of the chemical system. In various embodiments, the qubit representation may provide one or more reduced density matrices (RDMs) (e.g., such spinless (i.e., spin-traced) RDMs, such as a one particle RDM (1-RDM), 2 particle RDM (2-RDM), etc.) for the chemical system. In various embodiments, the qubit representation may provide a parameterized representation (e.g., an wavefunction) of the chemical system. In various embodiments, the qubit representation may be processed to determine one or more RDMs and/or other characteristics or properties of the chemical system.

The classical computing component 110 may then utilize and/or process the qubit representation of the chemical system to determine a wavefunction and/or structural, interaction, and/or other characteristics of the chemical system. For example, the classical computing entity utilizes and/or processes the qubit representation of the chemical system to determine an approximation to the characteristics of the eigenstates of the total electronic Hamiltonian of the system, such as expectation values of quantum operators acting on such states, to complete the simulation of the chemical system, determine how the chemical system behaves in one or more interactions (e.g., with other chemical systems and/or with electromagnetic radiation), determine a structural characteristics of the chemical system, and/or the like.

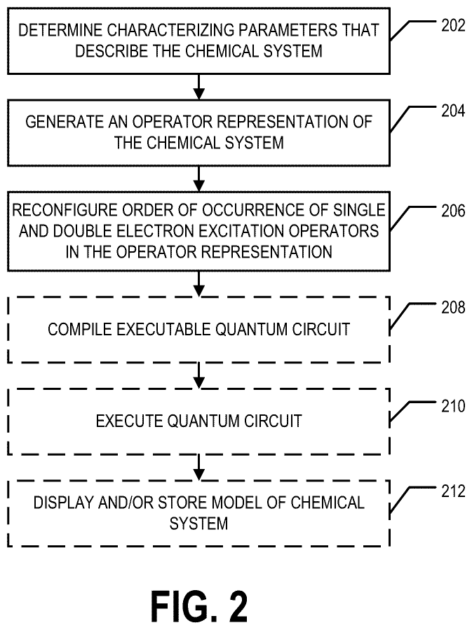

FIG. 2 provides a flowchart of various processes, procedures, operations, and/or the like performed by the hybrid quantum-classical computing system 100, in various embodiments. Starting at step/operation 202 of FIG. 2, the classical computing component 110 determines characterizing parameters that describe the chemical system. For example, the classical computing component 110 may receive (e.g., through user input) information identifying and/or corresponding to the chemical system. For example, the classical computing component 110 may receive a chemical formula for the chemical system (H, H₂O, NH₄⁺, OH, CH₄, a specified transition state of a chemical reaction, etc.) and/or other designation of the chemical system. In an example embodiment, the information corresponding to the chemical system includes nuclei information for each atomic nucleus of the chemical system (e.g., numbers of protons and neutrons present in the respective atomic nuclei), a number of electrons in the chemical system, Cartesian coordinates of the nuclei, and/or any other information used to define the chemical system.

Based on the information identifying and/or corresponding to the chemical system, the classical computing component 110 determines characterizing parameters of the chemical system. In various embodiments, the characterizing parameters are determined at least in part based on the chemical structure of the chemical system. In various embodiments, the characterizing parameters are determined using one or more of look-up tables relating to the structure of one or more chemical systems, processing information corresponding to a geometry of the chemical system (e.g., the Cartesian coordinates of the nuclei), determining one or more orbitals of the chemical system using a Hartree-Fock technique and/or perturbation theory technique, and/or the like.

For example, the characterizing parameters include nuclei information for each atomic nucleus of the chemical system (e.g., numbers of protons and neutrons present in the respective atomic nuclei), a number of electrons in the chemical system, Cartesian coordinates of the nuclei, various symmetries of the chemical system (e.g., including but not limited to [symbol] ₂ symmetries), spatial and/or spin orbitals of the chemical system (e.g., approximated via Hartree-Fock (HF) techniques, and/or the like), and/or other parameters that characterize the chemical system based at least in part on the structure of the chemical system, in various embodiments.

At step/operation 204, the classical computing component 110 generates a parameterized representation and/or operator representation of the chemical system. For example, an ansatz is identified and/or selected (e.g., UCCSD and/or another appropriate ansatz) and a corresponding operator representation representing the electron correlation of the chemical system is determined based on the identified and/or selected ansatz. In various embodiments, the ansatz is identified based on user input (e.g., received via a user input device), selected based on a default ansatz, and/or the like.

In various embodiments, the operator representation of the chemical system comprises an operator that describes the electron correlations of the chemical system. For example, in various embodiments, the operator representation of the chemical system includes an operator that includes a sum over each excitation operator acting across all of the orbitals of the chemical system. For example, the operator representation of the chemical system is an aggregated operator formed by summing over each excitation operator acting across all of the spin and/or spatial orbitals of the chemical system. In various embodiments, the excitation operators included in the operator representation of the chemical system are determined based at least in part on one or more of spin-multiplicity of the chemical system, number of electrons in the chemical system, and the basis-set size of the chemical system.

In various embodiments, a parameterized representation of the chemical system is determined based on the operator representation of the chemical system. For example, in various embodiments, the parameterized representation is a wavefunction of the chemical system and the operator representation is an effective Hamiltonian of at least a portion of the chemical system. In various embodiments, the parameterization of the parameterized representation and/or operator representation of the chemical system is determined based on an ansatz to be used to represent the waveform of the chemical system and/or operators of the chemical system. In an example embodiment, the ansatz used to represent the waveform of the chemical system and/or operators of the chemical system is a unitary coupled cluster including single electron excitation operators and double electron excitation operators (UCCSD) ansatz.

In various embodiments, the electron correlations of the chemical system are modelled and/or simulated using an operator representation of the chemical system which is and/or includes an ansatz-determined operator. For example, in various embodiments using a UCCSD ansatz, the electron correlations are simulated by using a UCCSD operator. For example, in an example embodiment, the operator representation of the chemical system is a UCCSD operator or another ansatz-determined operator.

In an example embodiment, the operator representation includes a UCCSD operator that is represented as a Trotterized product of exponentials. As is generally understood in the art, Trotterizing a product of exponents is a truncation of the infinite series used to evaluate the product of exponentials. For example, in an example embodiment, the UCCSD operator is written as e^{\hat{⬆}-\hat{⬆}†} where \hat{T} is the sum over each excitation operator acting across all of the orbitals of the chemical system and \hat{T}^{†} is the Hermitian adjoint thereof. For example, in various embodiments, the operator representation of the chemical system comprises an effective Hamiltonian of the chemical system.

In an example embodiment, the operator representation of the chemical system (e.g., the UCCSD operator, in an example embodiment) is transformed and/or mapped from the Hilbert space of the chemical system to the Hilbert space of the qubits of the quantum computing component. In various embodiments, the transformation and/or mapping of the operator representation of the chemical system from fermionic operators to qubit operators is determined and/or dependent on the type of qubit used by the quantum component of the hybrid quantum-classical computing system being used. For example, the quantum component may use photons, electrons, atomic nuclei, neutral atoms, ions, Josephson junctions, quantum dots, topological anyons, and/or other quantum particles and/or systems as qubits. In an example embodiment, the transformation and/or mapping is performed using a Jordan-Wigner encoding, although various other encodings are used in various other embodiments.

In embodiments using a Jordan-Wigner encoding, for example, each qubit of the quantum circuit represents the electron number occupation of each spin-orbital in the chemical system. For example, when a qubit is in the |1> state, the corresponding spin-orbital is occupied and when the qubit is in the |0> state, the corresponding spin-orbital is not occupied. Using the Jordan-Wigner encoding, the operator representation (e.g., including the UCCSD operator or other ansatz-determined operator) is written as a product of Pauli exponentials such that e^{\hat{⬆}-\hat{⬆}†}≈Πₘ Πₙ e^{iθ}^{ₘ}^{\hat{P}}^{_{m,n}}, where the index m runs over each distinct Fermionic excitation of the chemical system, the index n runs over each Pauli-sub-term \hat{P}_{m,n} of the Pauli operator \hat{P}, and the θₘ are independent real parameters.

At step/operation 206, the classical computing component 110 reconfigures the order of occurrence of single and double electron excitation operators in the operator representation of the chemical system. The reconfiguration reduces the number and/or lowers the count of two-qubit quantum gates (e.g., two qubit gates) used in the quantum circuit to model and/or simulate the chemical system. In various embodiments, the reconfiguration of the occurrence of the single electron excitation operators and the double electron excitation operators is performed as a function of the characterizing parameters of the chemical system. For example, the knowledge of the symmetry of the chemical system, spin and/or spatial orbitals of the chemical system, and/or the like are used to reconfigure the order of the occurrence of the single electron excitation operators and the double electron excitation operators in the operator representation of the chemical system so as to lower the count of two-qubit quantum gates in the resulting quantum circuit.

In various embodiments, the reconfiguration of the order of occurrence of the single electron excitation operators and the double electron excitation operators in the operator representation of the chemical system is performed based at least in part on the characterizing parameters of the chemical system. For example, the reconfiguration of the occurrence of the single electron excitation operators and the double electron excitation operators in the operator representation of the chemical system is performed based on a filtering of the excitation operators based on one or more symmetries of the chemical system, types of excitations (spatial orbital to spatial orbital double excitations, arbitrary double excitations, arbitrary single excitations), through the definition of a (double occupation of spatial orbitals) reference state, transforming at least some of the spatial orbitals to spin orbitals, commuting sets of electron excitation operators, and/or the like.

In various embodiments, reconfiguring the order of occurrence of the single electron excitation operators and the double electron excitation operators in the operator representation of the chemical system includes applying symmetry filtering to the waveform representation of the chemical system to assess its structural degree of symmetry.

For example, in an example embodiment, the classical computing component 110 applies the symmetry filtering by defining a set of symmetries of the chemical system and identifying one or more excitation operators of the operator representation of the chemical system that do not commute with one or more symmetries of the set of symmetries. The one or more excitation operators that do not commute with one or more symmetries of the set of symmetries are removed from the operator representation of the chemical system. In an example embodiment, the set of symmetries is the set of [symbol] ₂ symmetries of the chemical system.

In an example embodiment, the classical computing component 110 applies the symmetry filtering by defining an abelian point group of the chemical system and identifying redundant excitations of the chemical system based on the abelian point group. For example, the largest and/or highest abelian point group of the chemical system is defined and used to identify redundant excitations of the chemical system, in an example embodiment. The excitation operators corresponding to the excitations of the chemical system identified as being redundant excitations are removed from the operator representation of the chemical system.

Conventionally, in the Trotterized operator representation (e.g., in a UCCSD operator thereof, in an example embodiment), single electron excitation operators are applied prior to the application of double electron excitation operators. According to various embodiments, the order of occurrence of the excitation operators within the operator representation of the chemical system is reconfigured such that double electron excitation operators corresponding to pairs of electrons being excited from one spatial orbital to another spatial orbital \hat{T}^{e} are applied first. Arbitrary double electron excitation operators Td (e.g., double electron excitation operators that correspond to excitations other than a pair of electrons being excited from one spatial orbital to another spatial orbital) are applied after the application of the double electron excitation operators from one spatial orbital to another spatial orbital \hat{T}^{e}. Arbitrary single electron excitation operators \hat{T}^{s} are applied after the application of the arbitrary double electron excitation operators Td.

For example, in various embodiments, the classical computing component 110 reconfigures the order of occurrence of the excitation operators within the operator representation of the chemical system so that the effective Hamiltonian, in embodiments using an UCCSD ansatz, is of the form

$${Π}_{n} {e}^{{\hat{T}}_{n}^{s} -} {\hat{T}}_{n}^{{s}^{†}} {Π}_{k} {e}^{{\hat{T}}_{k}^{d} -} {\hat{T}}_{k}^{{d}^{†}} {Π}_{m} {e}^{{\hat{T}}_{m}^{e} -} {\hat{T}}_{m}^{{e}^{†}} .$$

In various embodiments, reconfiguring the order of occurrence of the single electron excitation operators and the double electron excitation operators in the operator representation of the chemical system includes generating a quantum state synthesis of the chemical system based on the degree of symmetry of the chemical system. In various embodiments, generating the quantum state synthesis comprises generating a double occupation of spatial orbitals reference state by processing a reference HF state of the chemical system to extract spatial orbital occupations. In an example embodiment, only the double occupied spatial orbitals and the virtual spatial orbitals are included in the double occupation of spatial orbitals reference state. For example, for a HF state of |110000> defining the spin orbital occupation (where the first two l's indicate a pair of occupied spin orbitals corresponding to a same spatial orbital and the four 0's correspond to two pairs of unoccupied virtual spin orbitals of the chemical system) would be processed to provide the double occupation of spatial orbitals reference state |100>, indicating that the first spatial orbital is doubly occupied and the second and third spatial orbitals are unoccupied. In an example embodiment, these spatial orbital occupations are mapped to even-indexed qubits of the quantum circuit by applying Pauli-X gates to provide the qubit state |100000>. In an example embodiment, the quantum state synthesis causes the qubits (e.g., the even-indexed qubits) to encode the (double) occupation of the spatial orbitals of the chemical system, rather than the occupation of the spin orbitals of the chemical system.

In various embodiments, reconfiguring the order of occurrence of the single electron excitation operators and the double electron excitation operators in the operator representation of the chemical system includes generating a spatial excitation synthesis of the chemical system from the quantum state synthesis. In various embodiments, double electron spatial orbital to spatial orbital operators enact operations that excite a pair of electrons from a first spatial orbital to a second spatial orbital. As the pair of electrons is excited together (e.g., as a pair), the pair of electrons can be approximated as a hard-core boson. In other words, the double electron excitation operators corresponding to pairs of electrons being excited from one spatial orbital to another spatial orbital \hat{T}^{e} can be expressed as hard-core bosonic operators acting on the spatial orbitals of the chemical system. As should be understood, hard-core bosons are particles within integer spin quantum numbers and that cannot occupy the same quantum state. For example, the occupation of a spatial orbital by hard-core bosons comprising pairs of electrons is either 0 or 1. In various embodiments, the action of each hard-core bosonic operators can be implemented using two two-qubit quantum gates, whereas the corresponding fermionic operator based operation is implement using four two-qubit quantum gates.

In various embodiments, reconfiguring the order of occurrence of the single electron excitation operators and the double electron excitation operators in the operator representation of the chemical system includes performing a change of representation so that the qubits of the quantum circuit carry spin orbital occupation information. For example, the qubits corresponding to the spin orbitals that are part of the doubly occupied spatial orbitals are each caused to indicate that the spin orbitals are occupied. The qubits corresponding to the spin orbitals that are part of unoccupied spatial orbitals are caused to indicate that the spin orbitals are not occupied. Qubits corresponding to spin orbitals that are part of singly occupied spatial orbitals may be toggled to indicate the appropriate single occupation of the respective spatial orbital, as appropriate.

In various embodiments, reconfiguring the order of occurrence of the single electron excitation operators and the double electron excitation operators in the operator representation of the chemical system includes generating a quantum circuit synthesis by using commuting sets of operators. In various embodiments, each commuting set of operators represents an excitation. The operators of the commuting set of operators are configured to act on and/or in the qubit Hilbert space for qubits of a quantum computing component to be used to execute the resulting at least one quantum circuit. In various embodiments, the commuting sets are generated and/or determined using tket. For example, in various embodiments, the commuting sets technique may be similar to and/or use the framework disclosed by U.S. Pat. No. 11,144,689, issued Oct. 12, 2021, the content of which is incorporated herein by reference in its entirety.

For example, the quantum circuit synthesis is generated, in part, by commuting sets of double electron excitation operators from the processed spatial excitation synthesis. For example, the spatial excitation synthesis results in a quantum circuit fragment that corresponds to application of the double electron excitation operators corresponding to pairs of electrons being excited from one spatial orbital to another spatial orbital \hat{T}^{e}. The quantum circuit fragment is then built upon to include the application of the arbitrary double electron excitation operators \hat{T}^{d}. In various embodiments, the application of the arbitrary double electron excitation operators \hat{T}^{d} is added to the quantum circuit using a commuting sets technique.

For example, in an example embodiment, the minimum number of mutually commuting sets of arbitrary double electron excitation operators that accounts for all of the arbitrary double electron excitation operators \hat{T}_{d} is determined and appropriate circuit pieces for each mutually commuting set is determined and/or generated. The circuit pieces are added to the quantum circuit fragment corresponding to the application of the double electron excitation operators corresponding to pairs of electrons being excited from one spatial orbital to another spatial orbital \hat{T}^{e}.

In various embodiments, reconfiguring the order of occurrence of the single electron excitation operators and the double electron excitation operators in the operator representation of the chemical system includes amending the quantum circuit synthesis by commuting sets of singles, such that double excitation operators appear in the quantum circuit synthesis before the single excitation operators appear in the quantum circuit synthesis. For example, in various embodiments, the application of the arbitrary single electron excitation operators \hat{T}^{s} is added to the quantum circuit using a commuting sets technique. For example, in an example embodiment, the minimum number of mutually commuting sets of arbitrary single electron excitation operators that accounts for all of the arbitrary single electron excitation operators is determined and appropriate circuit pieces for each mutually commuting set is determined and/or generated. The circuit pieces are added to the quantum circuit corresponding to the application of the double electron excitation operators corresponding to pairs of electrons being excited from one spatial orbital to another spatial orbital \hat{T}^{e} and the arbitrary double electron excitation operators \hat{T}^{d}.

In various embodiments, reconfiguring the order of occurrence of the single electron excitation operators and the double electron excitation operators in the operator representation of the chemical system results in a quantum circuit that, when executed by a quantum computing component 130, causes the quantum computing component 130 to simulate the chemical system. Moreover, the resulting quantum circuit includes a lower count of two-qubit quantum gates, such as controlled-NOT (CX) gates, for example, than conventional quantum circuit synthesizing techniques.

At step/operation 208, the classical computing component 110 or the controller 132 of the quantum computing component 130 compiles the quantum circuit to generate a compiled and/or executable quantum circuit that is executable by the quantum computing component 130. For example, the compiled and/or executable quantum circuit comprises a set of commands and/or the like that are executable by the controller 132 to cause the controller 132 to control various components of the quantum computing component 130 to cause the quantum computing component 130 to execute the quantum circuit. In an example embodiment, the quantum circuit is compiled using tket, though various other quantum circuit compilers may be used in various other embodiments.

At step/operation 210, the hybrid quantum-classical computing system 100 executes the compiled and/or executable quantum circuit. For example, the controller 132 controls various components of the quantum computing component 130 (e.g., the qubit manipulation elements 136 and/or sensors 138) to cause the quantum states of the qubits 134 to be evolved in a controlled manner such that measurements taken as part of the execution of the compiled and/or executable quantum circuit provide a qubit representation of the chemical system.

In various embodiments, executing the compiled and/or executable quantum circuit comprises measuring the quantum state of one or more qubits to determine a qubit representation of the chemical system. In various embodiments, the controller 132 and/or the classical computing component 110 processes the qubit representation of the chemical system to determine at least one of a wavefunction of the chemical system, structural characteristic of the chemical system, a chemical interaction characteristic of the chemical system, or a response characteristic of the chemical system. For example, the controller 132 and/or the classical computing component 110 performs a post-processing of the model and/or simulation of the chemical system encoded by the qubit representation of the chemical system to determine at least one of a wavefunction of the chemical system, structural characteristic of the chemical system, a chemical interaction characteristic of the chemical system, or a response characteristic of the chemical system. In various embodiments, the format or structure of the wavefunction corresponds to the ansatz used in the generation of the parameterized representation and/or the operator representation of the chemical system.

In various embodiments, a structural characteristic of the chemical system characterizes and/or provides information regarding the structure of the chemical system. For example, the structural characteristic may indicate a shape of the chemical system, an ordering and/or spatial distribution of the component atoms or atom groups within the chemical system, a characterization of various bonds of the chemical system, relative nuclei positions within the chemical system, and/or the like.

In various embodiments, a chemical interaction characteristic of the chemical system characterizes and/or provides information regarding how the chemical system interacts with one or more other chemical systems of the same or different types. For example, the chemical interaction characteristic of the chemical system may provide information regarding how the chemical system interacts with other chemical systems of the same type/chemical formula as the chemical system or of different types/chemical formulas than that of the chemical system.

For example, the structural and/or chemical interaction characteristic may be a dissociation curve, bond energy, reaction energy, reaction barrier, binding energy, adsorption energy and/or the like.

In various embodiments, the response characteristics of the chemical system characterize and/or provide information regarding how the chemical system interacts with electromagnetic radiation. For example, a response characteristic of the chemical system may be an excitation energy, singlet-triplet gap, photodissociation energy, photoionization energy, absorption cross-section, dielectric constant, dielectric function, oscillator strength and/or the like.

At step/operation 212, the classical computing component 110 provides at least a portion of the model and/or simulation of the chemical system representing the wavefunction, structural characteristic, the chemical interaction characteristic, and/or the response characteristic of the chemical system. In various embodiments, providing the at least a portion of the model and/or simulation of the chemical system comprises displaying, storing, transmitting (e.g., via one or more wired and/or wireless networks), providing a call response (e.g., application program interface (API) call response), and/or the like.

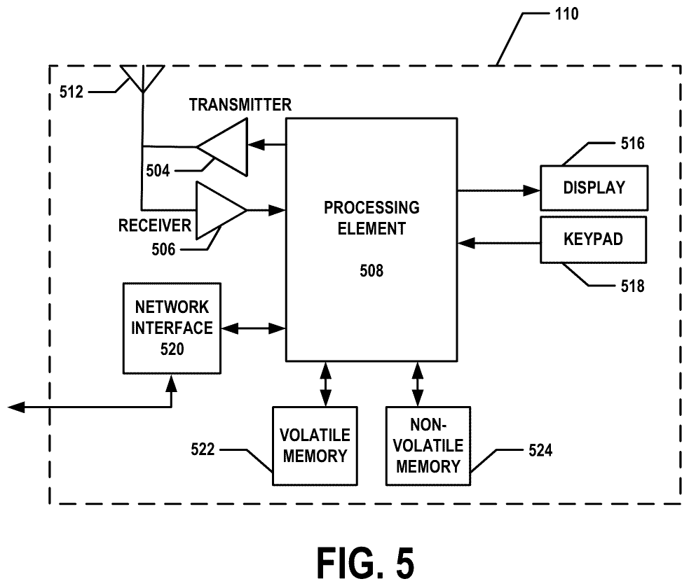

For example, in an example embodiment, the classical computing component 110 causes a display (e.g., display 516 illustrated in FIG. 5 and/or another display) to display a representation of the model and/or simulation of the chemical system. For example, a graphics processing unit (GPU) of the classical computing component 110 may generate a graphical representation of at least a portion of the model and/or simulation of the chemical system that, for example, provides a visualization of the wavefunction, structural characteristic, chemical interaction characteristic, and/or response characteristic of the chemical system. The classical computing component 110 may then cause the graphical representation of the at least a portion of the model and/or simulation of the chemical system to be displayed via a display for review and/or viewing by a human user.

In another example, the classical computing component 110 may generate and store (e.g., in memory 522, 524) a file comprising at least a portion of the model and/or simulation of the chemical system. For example, the file may comprise the wavefunction, the structural characteristic, the chemical interaction characteristic, the response characteristic, the chemical formula of the chemical system, and/or the like. The file may then be provided to one or more programs, applications, modules, and/or the like operating on the classical computing component 110 or another computing entity as input for one or more functions and/or computations performed thereby. For example, the file storing and/or encoding the at least a portion of the model and/or simulation of the chemical system may be used by various programs, applications, modules, and/or the like to generate graphical representations and/or visualizations of the chemical system and/or portions thereof, perform simulations that include the interaction of the chemical system with one or more other chemical systems (of the same of different chemical formulas), perform simulations of a bulk material that includes the chemical system, perform simulations that include the interaction of the chemical system with one or more biological systems, perform simulations to determine optical spectra of the chemical system, and/or the like.

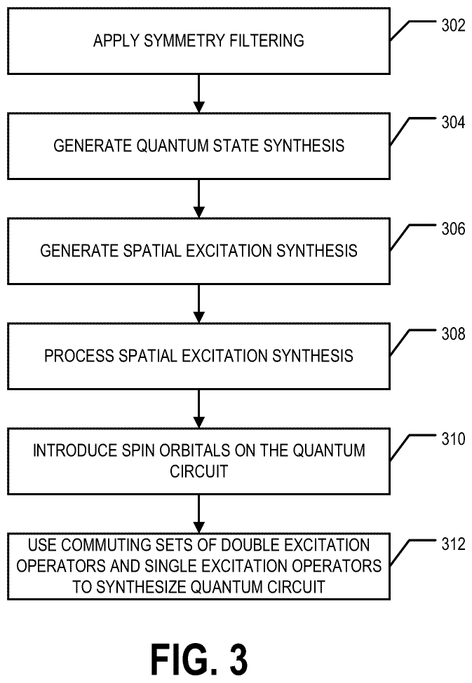

FIG. 3 provides a flowchart illustrating various processes, procedures, operations, and/or the like performed by the hybrid quantum-classical computing system 100 to reconfigure the order of occurrence of the single electron excitation operators and the double electron excitation operators in the operator representation of the chemical system. For example, the steps/operations of the flowchart shown in FIG. 3 are performed as part of step/operation 208, in an example embodiment. As noted elsewhere herein, the operator representation of the chemical system includes an effective Hamiltonian for the chemical system (or for at least a portion of the electrons of the chemical system). The order in which the operators that comprise the effective Hamiltonian are ordered and/or represented (e.g., within a quantum circuit) effects the number of two-qubit quantum gates required to implement the modeling and/or simulation of the chemical system via the quantum circuit to be executed by a quantum computing component 130.

In various embodiments, the symmetry filtering of the excitation operators and/or the treatment of double electron spatial orbital to spatial orbital excitations as bosonic excitations and the corresponding qubit reference frame transformations enable the modeling and/or simulating of a chemical system via a quantum circuit that requires significantly fewer (e.g., a factor of ten or more) and/or a lower count of two-qubit quantum gates (e.g., two qubit gates such as the CX gate) compared to conventional quantum circuits for modeling and/or simulating the chemical system.

Starting at step/operation 302, the classical computing component 110 applies symmetry filtering. The symmetry filtering removes and/or filters out excitation operators from the operator representation of the chemical system that are not allowed and/or that are redundant based on the symmetry of the chemical system. This reduces the overall number of excitation operators present in the operator representation of the chemical system.

For example, in various embodiments, applying the symmetry filtering comprises filtering the set of excitations present in the operator representation of the chemical system using symmetry of the chemical system (e.g., molecular symmetry and/or the like) to identify forbidden terms in the operator representation. In various embodiments, a first symmetry filtering is performed. The first symmetry filtering includes defining a set of symmetries of the chemical system (e.g., the set of [symbol] ₂ symmetries) and checking the commutation of the excitation operators of the operator representation of the chemical system against the set of symmetries to identify and discard forbidden excitations. In various embodiments, a second symmetry filtering is performed. The second symmetry filtering includes determining and/or identifying a highest Abelian point group of the chemical system and using the highest Abelian point group of the chemical system to identify and discard redundant excitations. In various embodiments, both the first symmetry filtering and the second symmetry filtering are performed.

In various embodiments, the symmetry filtering is performed and/or applied by identifying and/or defining symmetries of the chemical system. In various embodiments, the symmetries of the chemical system are identified and/or defined at least in part based on the characterizing parameters of the chemical system. For example, the geometry of the chemical system, as indicated by the characterizing parameters of the chemical system may be processed to identify and/or determine symmetries of the chemical system.

For example, a set of symmetries and/or a point group having the same symmetries as the chemical system is identified. The identified set of symmetries and/or point group corresponds to a degree of symmetry of the chemical system. Based on the identified set of symmetries and/or point group, excitation operators that enact and/or correspond to excitations that are not allowed excitations and/or excitations that are redundant are removed from the excitation operators present in the operator representation of the chemical system. In other words, the excitation operators identified as enacting and/or corresponding to excitations that are not allowed and/or that are redundant are removed from the operator representation of the chemical system, thereby reducing the number of operations included therein.

For example, in an example embodiment, the classical computing component 110 applies the first symmetry filtering by defining a set of symmetries of the chemical system and identifying one or more excitation operators of the operator representation of the chemical system that do not commute with one or more symmetries of the set of symmetries. The one or more excitation operators that do not commute with one or more symmetries of the set of symmetries enact and/or correspond to excitations that are not allowed. Thus, the one or more excitation operators that do not commute with one or more symmetries of the set of symmetries are removed from the operator representation of the chemical system. In an example embodiment, the set of symmetries is the set of [symbol] ₂ symmetries of the chemical system. As should be understood, the set of [symbol] ₂ symmetries corresponds to the symmetries of the set −1 and 1 with a multiplicative operator and/or the set 0 and 1 with the addition mod 2 operator. For example, the first symmetry filtering may be performed similar to the manner described by “Reducing qubit requirements for quantum simulation using molecular point group symmetries” by Kanav Setia, et al. and/or “Momentum-space unitary coupled cluster and translational quantum subspace expansion for periodic systems on quantum computers” by David Zsolt Marique, et al., in various embodiments.

In an example embodiment, the classical computing component 110 applies the second symmetry filtering by defining an abelian point group of the chemical system and identifying redundant excitations of the chemical system based on the abelian point group. For example, the largest and/or highest abelian point group of the chemical system is defined and used to identify redundant excitations of the chemical system, in an example embodiment. The excitation operators corresponding to the excitations of the chemical system identified as being redundant excitations are removed from the operator representation of the chemical system. For example, the second symmetry filtering may be performed similar to the manner described by “Use of molecular symmetry in coupled-cluster theory” by Petr Čársky, et al., in various embodiments.

In various embodiments, the symmetry filtering includes only one of (a) defining a set of symmetries of the chemical system and identifying not allowed excitations based thereon or (b) defining the largest abelian group of the chemical system and identifying redundant excitations based thereon. In various embodiments, the symmetry filtering includes both (a) defining a set of symmetries of the chemical system and identifying not allowed excitations based thereon and (b) defining the largest abelian group of the chemical system and identifying redundant excitations based thereon.

After performing and/or applying the symmetry filtering, the remaining excitation operators of the operator representation of the chemical system are processed to generate and/or synthesize a quantum circuit for modelling and/or simulating the chemical system. For example, the quantum circuit includes preparing a set of qubits to encode a spin orbital occupation of the chemical system through the application of the effective Hamiltonian of the chemical system (or the effective Hamiltonian for at least some of the electrons of the chemical system) through a transformation of the effective Hamiltonian to qubit operators effectible via quantum gates enacted on the corresponding qubits.

In various embodiments, the quantum circuit is synthesized, via steps/operations 304-312, such that the qubit operators that are the transformed and/or mapped representation and/or version of the double electron spatial orbital to spatial orbital excitation operators appear in the quantum circuit prior to the qubit operators that are the transformed and/or mapped representation and/or version of the arbitrary double electron excitation operators and such that the qubit operators that are the transformed and/or mapped representation and/or version of the arbitrary double electron excitation operators appear in the quantum circuit prior to the qubit operators that are the transformed and/or mapped representation and/or version of the arbitrary single electron excitation operators. In an example embodiment, at least a portion of steps/operations 304-312 are performed by the classical computing component 110 using tket or another quantum circuit generation and/or compiling program.

At step/operation 304, the classical computing component 110 generates the quantum state synthesis portion of the quantum circuit. In various embodiments, generating the quantum state synthesis comprises generating a (double occupation of spatial orbitals) reference state by processing a reference HF state of the chemical system to extract and/or determine reference spatial orbital occupations. For example, a reference HF state of the chemical system is provided by the characterizing parameters of the chemical system, in an example embodiment. The reference HF state is processed and/or analysed to identify spatial orbitals that are doubly occupied (e.g., both spin orbitals of the spatial orbitals are occupied) and/or virtual spatial orbitals that are not occupied. In an example embodiment, only the double occupied spatial orbitals and the virtual spatial orbitals are included in the (double occupation of spatial orbitals) reference state.

An initial portion of the quantum circuit is generated that initializes a set of qubits. Each qubit is indexed by an index p. In various embodiments, the qubits indexed by p=2q and p=2q+1, where q is an integer correspond to the two spin orbitals of a respective spatial orbital q. The initial portion of the quantum circuit is generated to include operations configured to cause, for qubits corresponding to doubly occupied spatial orbitals q, corresponding qubit indexed by p=2q to be initialized to state |1>, indicating double occupation of the corresponding spatial orbital and, for all other qubits, to be initialized to state |0>. For example, the initial portion of the quantum circuit may include initializing operations to cause the states of the qubits to be initialized based on the double occupation of the spatial orbitals as indicated by the reference HF state.

For example, for a HF state of |110000> defining the spin orbital occupation (where the first two l's indicate a pair occupied spin orbitals corresponding to a same spatial orbital and the four 0's correspond to two pairs of unoccupied virtual spin orbitals of the chemical system) would be processed to provide the double occupation of spatial orbitals reference state |100>, indicating that the first spatial orbital is doubly occupied and the second and third spatial orbitals are unoccupied. In an example embodiment, these spatial orbital occupations are mapped to even-indexed qubits (indexed by p=2q) of the quantum circuit by applying Pauli-X gates to provide the qubit state |100000>. In an example embodiment, the quantum state synthesis causes the qubits (e.g., the even-indexed qubits) to encode the (double) occupation of the spatial orbitals of the chemical system, rather than the occupation of the spin orbitals of the chemical system.

At step/operation 306, the classical computing component 110 generates the spatial excitation synthesis portion of the quantum circuit. In various embodiments, the operators that excite a pair of electrons from a first spatial orbital to a second spatial orbital are transformed from fermionic operators that act on individual spin orbitals to bosonic operators that act on a spatial orbital (e.g., corresponding to a pair of spin orbitals). As the pair of electrons is excited together (e.g., as a pair), the pair of electrons can be approximated as a hard-core boson (e.g., as an entity having integer spin). In other words, the double electron excitation operators corresponding to pairs of electrons being excited from one spatial orbital to another spatial orbital \hat{T}^{e} can be expressed as Hard-core bosonic operators acting on the spatial orbitals of the chemical system. In various embodiments, the action of each hard-core bosonic operator can be implemented using two two-qubit quantum gates, whereas the corresponding fermionic operator-based operation is implement using four two-qubit quantum gates.

Thus, the double electron spatial orbital to spatial orbital excitation operators \hat{T}^{e} of the operator representation (e.g., effective Hamiltonian) of the chemical system are transformed and/or mapped to hard-core bosonic operators. The resulting operators (e.g., the bosonic operators corresponding to the double electron spatial orbital to spatial orbital excitation operators of the operator representation) are then transformed and/or mapped to corresponding qubit operators that act on the even-indexed (e.g., p=2q) qubits. A second portion of the quantum circuit is generated that enacts the qubit operators that correspond to the bosonic operator-based application of the double electron spatial orbital to spatial orbital excitation operators \hat{T}^{e}. For example, the classical computing component 110 generates a second portion of the quantum circuit that corresponds to the application of the double electron spatial orbital to spatial orbital excitation operators \hat{T}^{e} using a hard-core bosonic operator representation thereof. In various embodiments, the second portion of the quantum circuit is generated using commuting sets of operators that are the transformed or mapped representations of the hard-core bosonic operators representing the double electron spatial orbital to spatial orbital excitation operators \hat{T}^{e}.

At step/operation 308, the classical computing component 110 processes the spatial excitation synthesis. The initial portion of the quantum circuit initialized the even-indexed qubits to represent the double occupation of spatial orbitals. The second portion of the quantum circuit performs excitation operations corresponding to exciting electrons from doubly occupied spatial orbitals to spatial orbitals that were previously unoccupied. The classical computing component 110 now generates a third portion of the quantum circuit that changes the representation of the qubits. For example, the third portion of the quantum circuit changes the semantic meaning of the states of the qubits such that each qubit corresponds to the occupation of a respective spin orbital.

For example, if the state of the qubit indexed by p=2q is |1> at the end of the second portion of the quantum circuit, then the corresponding spatial orbital q is doubly occupied and the third portion of the quantum circuit causes the qubit indexed by p=2q+1 to be set to state |1>, while maintaining the qubit indexed by p=2q in state |1> to indicate that both of the spin orbitals corresponding to the spatial orbital q are occupied.

Realistically, at the end of the second portion of the quantum circuit the state of the qubit indexed by p=2q is a super-position of the 11> and 10> states given by a|0>+b|1> where a²+b²=1. The third portion of the quantum circuit causes the qubit indexed by p=2q+1 to be in a same state as qubit indexed by p=2q, for qubits corresponding to spatial orbitals q that were doubly occupied or unoccupied in the HF reference state.

At step/operation 310, the classical computing component 110 introduces spin orbitals on the quantum circuit. For example, the classical computing component 110 generates a fourth portion of the quantum circuit that introduces the spin orbitals on the quantum circuit. The fourth portion of the quantum circuit initializes qubits corresponding to spatial orbitals that were singly occupied in the HF reference state. The initial, second, and third portions of the quantum circuit have not acted and/or performed quantum gates on the qubits corresponding to singly occupied spatial orbitals (according to the HF reference state) beyond initializing these qubits to the |0> state. The fourth portion of the quantum circuit includes operations that cause the state of qubits that correspond to the singly occupied spatial orbitals of the HF reference state to be toggled to state |1> to indicate the occupation of those spin orbitals.

At step/operation 312, the classical computing component 110 uses commuting sets of double excitation operators and single excitation operators (e.g., as provided by tket, in an example embodiment) to synthesis the quantum circuit. For example, the classical computing component 110 generates a quantum circuit synthesis including fifth and sixth portions of the quantum circuit. In various embodiments, each chemical excitation corresponds to a commuting set of operators. In various embodiments, a commuting set of operators is a set of Pauli operators that commute with one another and that are the transformed version of excitation operators from the operator representation of the chemical system.

For example, using commuting sets of operators, the classical computing component 110 generates a fifth portion of the quantum circuit that comprises qubit operators corresponding to the arbitrary double electron excitation operators \hat{T}^{d}. In various embodiments, the qubit operators corresponding to the arbitrary double electron excitation operators \hat{T}^{d} are Jordan-Wigner encoded arbitrary double excitation operators. In various embodiments, the Jordan-Wigner encoded arbitrary double excitation operators are configured to act on four or more qubits. Additionally, using commuting sets of operators, the classical computing component 110 generates a sixth portion of the quantum circuit that comprises qubit operators corresponding to the arbitrary single electron excitation operators Ts.

In various embodiments, the application of the arbitrary double electron excitation operators \hat{T}^{d} is added to the quantum circuit using a commuting sets technique. For example, in an example embodiment, the minimum number of mutually commuting sets of arbitrary double electron excitation operators that accounts for all of the arbitrary double electron excitation operators \hat{T}^{d} is determined and appropriate circuit pieces for each mutually commuting set is determined and/or generated. Each commuting set corresponds to a chemical excitation. These circuit pieces collectively form the fifth portion of the quantum circuit.

In various embodiments, the application of the arbitrary single electron excitation operators \hat{T}^{s} is added to the quantum circuit using a commuting sets technique. For example, in an example embodiment, the minimum number of mutually commuting sets of arbitrary single electron excitation operators that accounts for all of the arbitrary single electron excitation operators \hat{T}^{s} is determined and appropriate circuit pieces for each mutually commuting set is determined and/or generated. Each commuting set corresponds to a chemical excitation. These circuit pieces collectively form the sixth portion of the quantum circuit.

In various embodiments, the classical computing component 110 generates a seventh portion of the quantum circuit corresponding to performing measurement operations to measure the states of the qubits after the performance of the sixth portion of the quantum circuit. For example, the results of the measurements form and/or can be processed to form the qubit representation of the chemical system.

Thus, the classical computing component 110 generates a quantum circuit comprising the initial portion of the quantum circuit, the second portion of the quantum circuit, the third portion of the quantum circuit, the fourth portion of the quantum circuit, the fifth portion of the quantum circuit, the sixth portion of the quantum circuit, and, optionally, the seventh portion of the quantum circuit. The portions of the quantum circuit are ordered as listed here such that the qubit operations corresponding to the double electron spatial orbital to spatial orbital excitation operators are performed prior to the arbitrary double electron excitation operators, which are performed prior to the arbitrary single electron excitation operators.

### Technical Advantages

In general, it is expected that quantum computers will enable accurate modeling and/or simulation of chemical systems. For example, it is expected that quantum computers will enable the accurate modeling and/or simulation of chemical systems that are too complex for conventional classical approaches. However, currently operational quantum computing components, referred to as NISQ devices as aforementioned, tend to include relatively low numbers of qubits (e.g., less than 100 qubits) and tend to be relatively noisy. As the depth (e.g., the number of layers of quantum gats) and/or the number of two-qubit quantum gates of a quantum circuit increases, so does the effect of noise on the resulting model or simulation. For conventional techniques for generating quantum circuits to model and/or simulate a chemical system, the depth of the quantum circuit scales as O(n⁴), where n is the number of spin orbitals included in the model and/or simulation. These leads to quantum circuits that include thousands of two-qubit quantum gates, which is clearly beyond the capability of currently operable quantum computing components. Thus, a technical problem exists regarding how to generate quantum circuits for modeling and/or simulating chemical systems that can be executed by currently operable quantum computing components.

Various embodiments provide technical solutions to these technical problems. In particular, various embodiments use the symmetries of the chemical system to reduce the number of two-qubit quantum gates required to accurately model and/or simulate the chemical system. In various embodiments, the number of two-qubit quantum gates of the resulting quantum circuit is at least a factor of ten smaller than a quantum circuit for modeling and/or simulating the same chemical system generated through conventional techniques. Thus, various embodiments provide improvements to the fields of chemical system modeling and/or simulation, quantum chemistry, and/or quantum circuit generation.

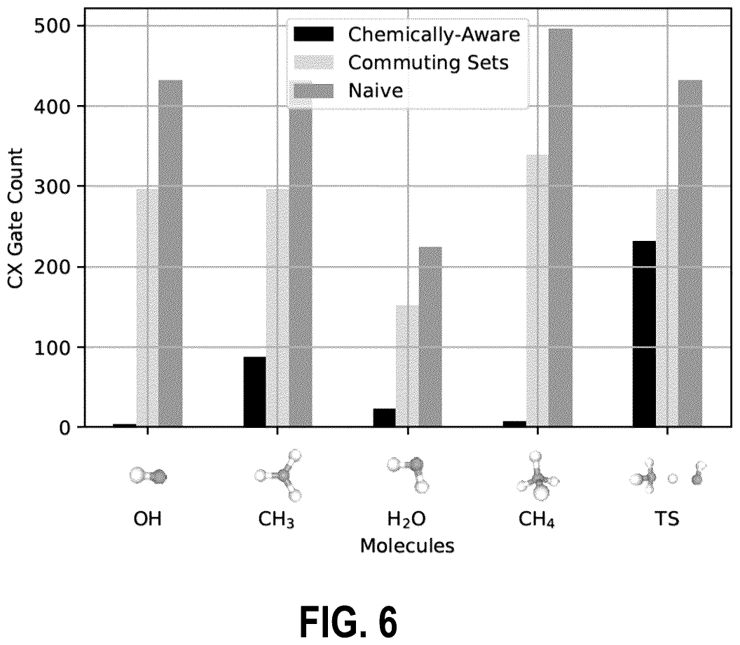

FIG. 6 provides an illustration of the controlled-NOT (CX) gate count of quantum circuits used to model and/or simulate various chemical systems (e.g., OH, CH₃, H₂O, CH₄, and the transition state (TS) of CH₃ and OH) for the chemically-aware quantum circuit synthesis of an example embodiment, a conventional commuting sets quantum circuit synthesis, and a conventional naïve quantum circuit synthesis. As can be seen in FIG. 6, the number of two-qubit quantum gates (in the illustrated example, CX gates) for each of the chemical systems is significantly reduced using the chemically-aware quantum circuit synthesis of an example embodiment compared to conventional quantum circuit synthesis techniques. Thus, as shown in FIG. 6, various embodiments provide improvements to the fields of chemical system modeling and/or simulation, quantum chemistry, and/or quantum circuit generation.

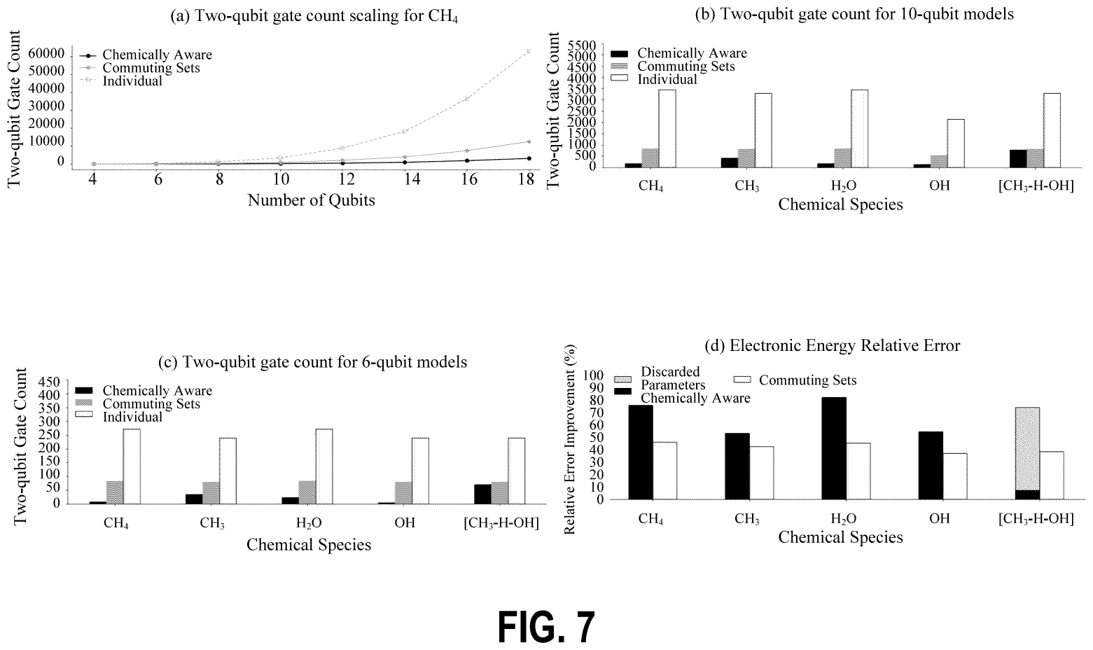

FIG. 7 provides a set of plots illustrating a comparison of the number of gates in quantum circuits and improvement in relative error on ground state energy determined based on the quantum circuits using an example embodiment of the present disclosure and using a commuting sets technique. Panel a of FIG. 7 illustrates the two-qubit gate count scaling for the chemical system CH₄ for various active spaces of the chemical system (e.g., for the number of qubits used to represent the active space) for an example embodiment (labelled as “chemically aware”), for a commuting sets technique, and for an individual technique where the operation representation of the chemical system is directly or naively transformed into a quantum circuit.

Panel b of FIG. 7 illustrates the two-qubit gate count comparison between an example embodiment of the present disclosure (labelled “Chemically Aware”), a commuting sets technique, and an individual synthesis quantum circuit for 10 qubit models. Panel c of FIG. 7 illustrates the two-qubit gate count comparison between an example embodiment of the present disclosure (labelled “Chemically Aware”), a commuting sets technique, and an individual synthesis quantum circuit for 6 qubit models. Panel d of FIG. 7 shows the improvement in relative error on ground-state energy computed on a noisy intermediate scale quantum (NISQ) era quantum computer using a quantum circuit generated through an example embodiment of the present disclosure (labelled “Chemically Aware”) and a quantum circuit generated through an individual synthesis technique.

### Exemplary Controller

In various embodiments, hybrid quantum-classical computing system 100 comprises a quantum computing component 130. The quantum computing component 130 is configured to perform various quantum computations and/or calculations via execution of one or more quantum circuits and/or algorithms. In various embodiments, the quantum computing component 130 is configured to control operation of one or more components of the quantum computing component 130 (e.g., qubit manipulation elements 136, sensors 138), receive sensor signals indicating measurements captured by sensors 138, and/or communicate with a classical computing component 110. The quantum computing component 130 optionally includes trapped-ion qubits, cryogenic-cooled Josephson junction qubits, or photonics qubits, but not limited thereto.

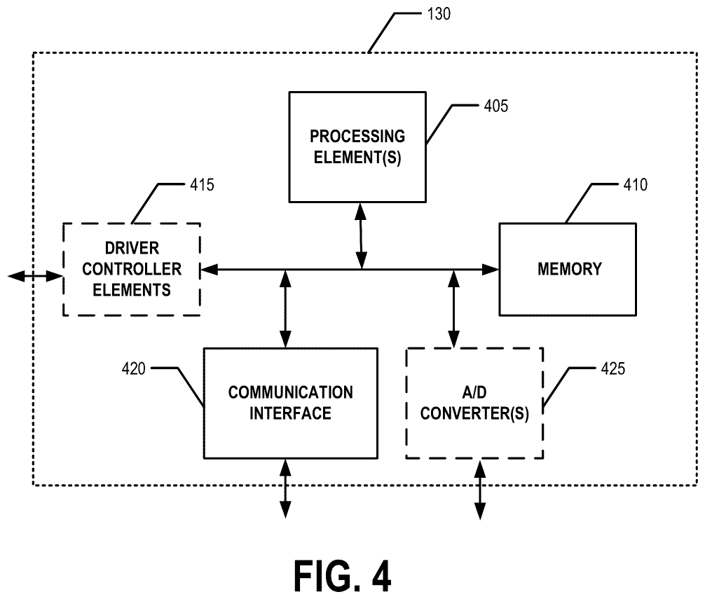

As shown in FIG. 4, in various embodiments, the controller 132 may comprise various controller elements including processing elements 405, memory 410, driver controller elements 415, a communication interface 420, analog-digital converter elements 425, and/or the like. For example, the processing elements 405 may comprise one or more processing devices such as programmable logic devices (CPLDs), microprocessors, coprocessing entities, application-specific instruction-set processors (ASIPs), integrated circuits, application specific integrated circuits (ASICs), field programmable gate arrays (FPGAs), programmable logic arrays (PLAs), hardware accelerators, other processing devices and/or circuitry, and/or the like. The term circuitry may refer to an entirely hardware embodiment or a combination of hardware and computer program products. In an example embodiment, the processing element 405 of the controller 132 comprises a clock and/or is in communication with a clock.

For example, the memory 410 may comprise non-transitory memory such as volatile and/or non-volatile memory storage such as one or more of as hard disks, ROM, PROM, EPROM, EEPROM, flash memory, MMCs, SD memory cards, Memory Sticks, CBRAM, PRAM, FeRAM, RRAM, SONOS, racetrack memory, RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. In various embodiments, the memory 410 may store a queue of commands to be executed to cause a quantum algorithm and/or circuit to be executed (e.g., an executable queue), qubit records corresponding the qubits of quantum computing component (e.g., in a qubit record data store, qubit record database, qubit record table, and/or the like), a calibration table, computer program code (e.g., in a one or more computer languages, specialized controller language(s), and/or the like), and/or the like. In an example embodiment, execution of at least a portion of the computer program code stored in the memory 410 (e.g., by a processing element 405) causes the controller 132 to perform one or more steps, operations, processes, procedures and/or the like described herein for controlling operation of one or more qubit manipulation elements 136, processing sensor signals indicating measurements captured by sensors 138, and/or communicating with a classical computing component 110 of the hybrid quantum-classical computing system 100.

In various embodiments, the driver controller elements 410 may include one or more drivers and/or controller elements each configured to control one or more drivers. In various embodiments, the driver controller elements 410 may comprise drivers and/or driver controllers. For example, the driver controllers may be configured to cause one or more corresponding drivers to be operated in accordance with executable instructions, commands, and/or the like scheduled and executed by the controller 132 (e.g., by the processing element 405). In various embodiments, the driver controller elements 415 may enable the controller 132 to operate various ones of the qubit manipulation elements 136 and/or sensors 138. In various embodiments, the drivers may comprise laser drivers configured to operate one or lasers; drivers for controlling operation of one or more voltage/current sources to cause generation and providing of one or more voltage and/or current signals; and/or various other drivers configured to control operation of respective qubit manipulation elements 136 of the quantum computing component 130.

In various embodiments, the controller 132 comprises means for communicating and/or receiving signals from one or more sensors (e.g., photodetectors, voltage/current sensors, temperature sensors, pressure sensors, and/or other sensors). For example, the controller 132 may comprise one or more analog-digital converter elements 425 configured to receive signals from one or more sensors.

In various embodiments, the controller 132 comprises a communication interface 420 for interfacing and/or communicating with a classical computing component 110 of the hybrid quantum-classical computing system 100. For example, the controller 132 may comprise a communication interface 420 for receiving one or more quantum circuits modeling and/or simulating a chemical system, executable instructions, command sets, and/or the like from the classical computing component 110 and providing output received from the quantum computing component 130 (e.g., via sensors 138) and/or the result of a processing the output to determine the qubit representation of the chemical system to the classical computing component 110. In various embodiments, the classical computing component 110 and the controller 132 may communicate via a direct wired and/or wireless connection and/or via one or more wired and/or wireless networks 120.

### Exemplary Classical Computing Component

FIG. 5 provides an illustrative schematic representative of an example computing entity 10 that can be used in conjunction with embodiments of the present invention. In various embodiments, a classical computing component 110 is configured to interface with a quantum computing component 130. For example, the classical computing component 110 is configured to interface with a quantum computing component 130 so as to enable the efficient and accurate modeling of a chemical system via execution of a quantum circuit having reduced depth and/or a lower count of a number of two-qubit quantum gates (compared to corresponding quantum circuits generated via conventional quantum circuit synthesis techniques) using the quantum computing component 130. For example, the classical computing component 110 may be configured to communicate with the quantum computing component 130 to allow a user (e.g., a human user or a program operating on the classical computing component 110) to provide input to the quantum computing component 130 and receive, display, analyze, and/or the like output from the quantum computing component 130.

As shown in FIG. 5, a classical computing component 110 can include an antenna 512, a transmitter 504 (e.g., radio), a receiver 506 (e.g., radio), and a processing element 508 that provides signals to and receives signals from the transmitter 504 and receiver 506, respectively. The signals provided to and received from the transmitter 504 and the receiver 506, respectively, may include signaling information/data in accordance with an air interface standard of applicable wireless systems to communicate with various entities, such as a controller 132, other classical computing entities 110, and/or the like. In this regard, the classical computing component 110 may be capable of operating with one or more air interface standards, communication protocols, modulation types, and access types.

For example, the classical computing component 110 may be configured to receive and/or provide communications using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the classical computing component 110 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra wideband (UWB), infrared (IR) protocols, near field communication (NFC) protocols, Wibree, Bluetooth protocols, wireless universal serial bus (USB) protocols, and/or any other wireless protocol. The classical computing component 110 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

Via these communication standards and protocols, the classical computing component 110 can communicate with various other entities using concepts such as Unstructured Supplementary Service information/data (USSD), Short Message Service (SMS), Multimedia Messaging Service (MIMS), Dual-Tone Multi-Frequency Signaling (DTMF), and/or Subscriber Identity Module Dialer (SIM dialer). The classical computing component 110 can also download changes, add-ons, and updates, for instance, to its firmware, software (e.g., including executable instructions, applications, program modules), and operating system.

In various embodiments, the classical computing component 110 may comprise a network interface 520 for interfacing and/or communicating with the controller 132, for example. For example, the classical computing component 110 may comprise a network interface 520 for providing qubit constraint information, executable instructions, command sets, and/or the like for receipt by the controller 132 and/or receiving output and/or the result of a processing the output (e.g., measured values corresponding to the active orbitals) provided by the quantum computing component 130. In various embodiments, the classical computing component 110 and the controller 132 may communicate via a direct wired and/or wireless connection and/or via one or more wired and/or wireless networks 120.

In various embodiments, the processing elements 508 may comprise one or more processing devices such as programmable logic devices (CPLDs), microprocessors, coprocessing entities, application-specific instruction-set processors (ASIPs), integrated circuits, application specific integrated circuits (ASICs), field programmable gate arrays (FPGAs), programmable logic arrays (PLAs), hardware accelerators, graphics processing units (GPUs), central processing units (CPUs), other processing devices and/or circuitry, and/or the like. The term circuitry may refer to an entirely hardware embodiment or a combination of hardware and computer program products.

The classical computing component 110 may also comprise a user interface device comprising one or more user input/output interfaces (e.g., a display 516 and/or speaker/speaker driver coupled to a processing element 508 and a touch screen, keyboard, mouse, and/or microphone coupled to a processing element 508). For instance, the user output interface may be configured to provide an application, browser, user interface, interface, dashboard, screen, webpage, page, and/or similar words used herein interchangeably executing on and/or accessible via the computing entity 10 to cause display or audible presentation of information/data and for interaction therewith via one or more user input interfaces. The user input interface can comprise any of a number of devices allowing the computing entity 10 to receive data, such as a keypad 518 (hard or soft), a touch display, mouse, voice/speech or motion interfaces, scanners, readers, or other input device. In embodiments including a keypad 518, the keypad 518 can include (or cause display of) the conventional numeric (0-9) and related keys (#, *), and other keys used for operating the classical computing component 110 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the classical computing component 110 can collect information/data, user interaction/input, and/or the like.

The classical computing component 110 can also include volatile storage or memory 522 and/or non-volatile storage or memory 524, which can be embedded and/or may be removable. For instance, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, data, applications, programs, program modules, scripts, source code, object code, byte code, compiled code, interpreted code, machine code, executable instructions, and/or the like to implement the functions of the classical computing component 110.

### CONCLUSION

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A hybrid quantum-classical computing system configured for implementing a simulation of a chemical system, wherein the hybrid quantum-classical computing system comprises a classical computing component coupled to a quantum computing component, wherein the hybrid quantum-classical computing system is configured to:
(i) determine characterizing parameters that describe the chemical system, wherein the characterizing parameters are at least partially based on a chemical structure of the chemical system;
(ii) generate an operator representation of the chemical system, including single electron excitation operators and double electron excitation operators; and
(iii) reconfigure an order of occurrence of the single electron excitation operators and the double electron excitation operators in the operator representation as a function of the characterizing parameters, such that the simulation is executable with a lower count of two-qubit quantum gates used in at least one quantum circuit executable on the quantum computing component that is used to implement the simulation.

2. The hybrid quantum-classical computing system of claim 1, wherein the hybrid quantum-classical computing system is configured to reconfigure the order of occurrence of the single electron excitation operators and the double electron excitation operators to:
(i) double excitation operators corresponding to double excitations from a spatial orbital to a corresponding spatial orbital;
(ii) arbitrary double excitation operators; and
(iii) arbitrary single excitation operators.

3. The hybrid quantum-classical computing system of claim 1, wherein the hybrid quantum-classical computing system is configured to determine the characterizing parameters that describe the chemical system and generate a corresponding operator representation:
(i) by applying symmetry filtering to the operator representation of the chemical system to assess its structural degree of symmetry;
(ii) by generating a quantum state synthesis of the chemical system based on the degree of symmetry;
(iii) by generating a spatial excitation synthesis of the chemical system from the quantum state synthesis using hard-core bosonic operators;
(iv) by processing the spatial excitation synthesis by transforming from spatial orbitals to spin orbitals therein;
(v) by introducing spin orbitals on the at least one quantum circuit; and
(vi) by using commuting sets of double excitation operators and single excitation operators to synthesize the at least one quantum circuit, such that double excitation operators appear in the at least one quantum circuit before the single excitation operators appear in the at least one quantum circuit and each commuting set corresponds to an excitation of the chemical system.

4. The hybrid quantum-classical computing system of claim 3, wherein the hybrid quantum-chemical computing system is configured to reduce a number of quantum gates required in the quantum circuit synthesis as a function of the structural degree of symmetry of the chemical system.

5. The hybrid quantum-classical computing system of claim 3, wherein applying the symmetry filtering to the operator representation comprises at least one of:
defining a set of symmetries of the chemical system and identifying excitation operators of the operator representation of the chemical system that do not commute with one or more symmetries of the set of symmetries, or
defining an abelian point group of the chemical system and identifying redundant excitations of the chemical system based on the abelian point group.

6. The hybrid quantum-classical computing system of claim 3, wherein generating the quantum state synthesis of the chemical system comprises generating a reference state based on the double occupied and virtual spatial orbitals of the chemical system.

7. The hybrid quantum-classical computing system of claim 3, wherein generating the spatial excitation synthesis comprises transforming spatial orbital to spatial orbital operators to double excitation spatial-to-spatial qubit operators.

8. The hybrid quantum-classical computing system of claim 7, wherein generating the quantum circuit synthesis comprises transforming the double excitation operators of the chemical system that correspond to double excitations other than spatial orbital to spatial orbital double excitations to Jordan-Wigner encoded arbitrary double excitation qubit operators configured to act on 4-qubits or more.

9. The hybrid quantum-classical computing system of claim 8, wherein the hybrid quantum-classical computing entity is configured to compile the quantum circuit synthesis into an executable quantum circuit, wherein the quantum circuit synthesis comprises, in order, the double excitation spatial-to-spatial qubit operators, the Jordan-Wigner encoded arbitrary double excitation qubit operators, and single excitation qubit operators.

10. The hybrid quantum-classical computing system of claim 1, wherein the hybrid quantum-classical computing system is further configured to cause the quantum computing component to execute the at least one quantum circuit to determine at least one of: a wavefunction of the chemical system, a structural characteristic of the chemical system, a chemical interaction characteristic of the chemical system, or a response characteristic of the chemical system.

11. The hybrid quantum-classical computing system of claim 10, wherein the hybrid quantum-classical computing system is further configured to cause the classical computing component to cause at least one of (a) display of a graphical representation of at least a portion of the simulation of the chemical system or (b) generation and storage in a classical memory of a file comprising one or more parameters of the simulation of the chemical system.

12. The hybrid quantum-classical computing system of claim 1, wherein the operator representation is generated using a unitary coupled cluster singles and doubles (UCCSD) ansatz.

13. A method for using a hybrid quantum-classical computing system to implement a simulation of a chemical system, wherein the hybrid quantum-classical computing system comprises a classical computing component coupled to a quantum computing component, wherein the method comprises:
(i) determining characterizing parameters that describe the chemical system, wherein the characterizing parameters are at least partially based on a chemical structure of the chemical system;
(ii) generating an operator representation of the chemical system, including single electron excitation operators and double electron excitation operators; and
(iii) reconfiguring an order of occurrence of the single electron excitation operators and the double electron excitation operators in the operator representation as a function of the characterizing parameters, such that the simulation is executable with a lower count of two-qubit quantum gates used in at least one quantum circuit executable on the quantum computing component that is used to implement the simulation.

14. The method of claim 13, wherein the order of occurrence of the single electron excitation operators and the double electron excitation operators is reconfigured to:
(i) double excitation operators corresponding to double excitations from a spatial orbital to a corresponding spatial orbital;
(ii) arbitrary double excitation operators; and
(iii) arbitrary single excitation operators.

15. The method of claim 13, wherein the system is configured to determine the characterizing parameters that describe the chemical system and generate a corresponding operator representation:
(i) by applying symmetry filtering to the operator representation of the chemical system to assess its structural degree of symmetry;
(ii) by generating a quantum state synthesis of the chemical system based on the degree of symmetry;
(iii) by generating a spatial excitation synthesis of the chemical system from the quantum state synthesis using hard-core bosonic operators;
(iv) by processing the spatial excitation synthesis by transforming from spatial orbitals to spin orbitals therein;
(v) by introducing spin orbitals on the at least one quantum circuit; and
(vi) by using commuting sets of double excitation operators and single excitation operators to synthesize the at least one quantum circuit, such that double excitation operators appear in the at least one quantum circuit before the single excitation operators appear in the at least one quantum circuit and each commuting set corresponds to an excitation of the chemical system.

16. The method of claim 15, wherein at least one of:
the hybrid quantum-chemical computing system is configured to reduce a number of controlled-NOT gates required in the quantum circuit synthesis as a function of the structural degree of symmetry of the chemical system,
applying the symmetry filtering to the operator representation comprises defining a set of symmetries of the chemical system and identifying excitation operators of the operator representation of the chemical system that do not commute with one or more symmetries of the set of symmetries,
applying the symmetry filtering to the operator representation comprises defining an abelian point group of the chemical system and identifying redundant excitations of the chemical system based on the abelian point group,
generating the quantum state synthesis of the chemical system comprises generating a reference state based on the double occupied and virtual spatial orbitals of the chemical system,
generating the spatial excitation synthesis comprises transforming spatial orbital to spatial orbital operators to double excitation spatial-to-spatial qubit operators, or
the quantum circuit synthesis comprises transforming the double excitation operators of the chemical system that correspond to double excitations other than double excitations from spatial orbital to spatial orbital to Jordan-Wigner encoded arbitrary double excitation qubit operators configured to act on 4-qubits or more.

17. The method of claim 16, further comprising compiling the quantum circuit synthesis into an executable quantum circuit, wherein the quantum circuit synthesis comprises, in order, the double excitation spatial-to-spatial qubit operators, the Jordan-Wigner encoded arbitrary double excitation qubit operators, and single excitation qubit operators.

18. The method of claim 13, further comprising:
executing, by the quantum computing component, the at least one quantum circuit to determine at least one of a wavefunction of the chemical system, a structural characteristic of the chemical system, a chemical interaction characteristic of the chemical system, or a response characteristic of the chemical system; and
causing, by the classical computing component, at least one of (a) display of a graphical representation of at least a portion of the model of the chemical system or (b) generation and storage in a classical memory of a file comprising one or more parameters of the model of the chemical system.

19. The method of claim 13, wherein the operator representation is generated using a unitary coupled cluster singles and doubles (UCCSD) ansatz.

20. A computer program product comprising at least one non-transitory computer-readable medium storing executable instructions, the executable instructions configured to, when executed by a hybrid quantum-classical computing system, cause the hybrid quantum-classical computing system to perform:
(i) determining characterizing parameters that describe the chemical system, wherein the characterizing parameters are at least partially based on a chemical structure of the chemical system;
(ii) generating an operator representation of the chemical system, including single electron excitation operators and double electron excitation operators; and
(iii) reconfiguring an order of occurrence of the single electron excitation operators and the double electron excitation operators in the operator representation as a function of the characterizing parameters, such that the simulation is executable with a lower count of two-qubit quantum gates used in at least one quantum circuit executable on the quantum computing component that is used to implement the simulation.
